(19) United States
(12) Patent Application Publication
(10) Pub. No.: US 2008/0226749 A1
(43) Pub. Date: Sep. 18, 2008

(54) Pharmaceutical composition for inhibiting the syndrome of snoring and preparation thereof

(75) Inventors: Shuenn-Jyi Sheu, Taipei City (TW); Hsiao-Yin Yeh, Taipei City (TW); Wu-Che Wen, Danshuei Township (TW); Ching-Che Lin, Taipei City (TW); Chen-Ying Chiang, Taipei City (TW)

Correspondence Address: BACON & THOMAS, PLLC, 625 SLATERS LANE, FOURTH FLOOR, ALEXANDRIA, VA, 22314-1176, US

(73) Assignee: Brion Research Institute of Taiwan, Taipei (TW)

(21) Appl. No.: 12/073,827

(22) Filed: Mar. 11, 2008

(30) Foreign Application Priority Data
Mar. 16, 2007 (TW) 096109064

(51) Int. Cl. A61K 33/06; A61K 36/00; A61P 11/00

(52) U.S. Cl. 424/696; 424/769

(57) ABSTRACT

The present invention discloses a pharmaceutical composition for inhibiting a snore symptom, which comprises medicinal material of Uncaria rhynchophylla. The above pharmaceutical composition of the present invention also comprises medicinal materials of Uncaria rhynchophylla, Ephedra, Prunus armeniaca, and Glycyrrhiza uralensis.

### BACKGROUND OF THE INVENTION

1. Field of the Invention

The present invention relates to a pharmaceutical composition, particularly to a pharmaceutical composition that inhibits or improves snoring of humans or animals.

2. Description of Related Art

### A. Causes of Snoring

The dangling flap of flesh located at the back of the throat is called the uvula. Snoring usually results from the narrow airway that leads to vibration of the uvula. Generally, the following factors contribute to snoring:
(a) lax muscles: the muscles around the uvula are lax due to aging and thereby the vibration of the uvula easily occurs;
(b) obesity: accumulation of fat leads to narrowing of the airway and thereby the vibration of the uvula easily occurs;
(c) sleeping pills, intoxication or fatigue result in deep sleep, and muscles are lax during deep sleep so that snoring occurs easily;
(d) smoking: increased excretions causes swelling of faucal mucosa, which narrows the airway and causes snoring;
(e) nasal congestion: allergic rhinitis, curling nasal septum, fleshy rhinitis, and nasosinusitis all aggravate snoring; and
(f) hypertrophy of tonsils (nasal or faucal tonsil hypertrophy, which is widely seen in children) also obstructs the airway and causes snoring;

### B. Sequela of Snoring: Obstructive Sleep Apnea

The definition of which is “5 or more pauses of breathing per hour during sleep, and each lasting above 10 seconds”. Generally, heavy snoring subjects suffer from pauses of breathing during sleep, obstructive breathing, awakening, low-quality sleeps, and thereby headache, dozing off or distraction during driving. The above symptoms are categorized as “Sleep Apnea Syndrome”. Long-term sleep apnea could lead to hypoxia and consequent damage to the heart and lungs, such as arrhythmia, coronary heart disease, hypertension, and other heart and lung diseases.

### C. Treatment of Snoring

There is no ideal pharmacotherapy to date, but physicians usually adopt conservative regimens first, urging patients to change their sleeping postures, lose weight, avoid smoking, intoxication, or medication (tranquilizers, antihistamine, etc.), in order to relieve the condition. If none of these remedies is successful, positive-pressure breathing apparatus or oral rectifiers are employed. However, surgical operations are more commonly used to treat snoring and sleep apnea.

There are two kinds of operation: traditional and laser operations. Traditional soft palate and uvula anaplasty is performed under general anaesthesia, cutting off the uvula and partial soft palate by electric burning or knife. The novel laser operations are performed using a carbon dioxide laser, which takes the advantages of the precision of the laser to cut off redundant oral and faucal tissues and expand oral and faucal cavity stage by stage, to improve snoring conditions.

### D. Uncaria Rhynchophylla

Uncaria rhynchophylla and plants of the same genus have a somewhat sweet flavor and are considered “cold” in nature from the point of view of Chinese medicine. It has the effects of tranquilizing, seizure inhibition, fever relieving, lowering blood pressure, and creating anti-viruses. Clinically, it is commonly used to treat fevers, seizures, acute infantile convulsions, liver fever, hypertension, night crying of babies, and epilepsy. However, there is no record to date that Uncaria rhynchophylla can inhibit snoring.

The present invention provides an Uncaria rhynchophylla material and a pharmaceutical composition comprising an Uncaria rhynchophylla material for improving or inhibiting snoring. Thus, when an Uncaria rhynchophylla material or the pharmaceutical composition thereof is utilized to improve snoring, the risks of surgical operations are avoided.

### SUMMARY OF THE INVENTION

The object of the present invention is to provide an Uncaria rhynchophylla material and the pharmaceutical composition thereof for improving or inhibiting animal snoring, and thereby preventing sleep apnea, headache, distraction, or fatigue caused by snoring and avoiding damages caused by hypoxia to the heart and lungs.

To achieve the object, the present invention provides a pharmaceutical composition for inhibiting snoring, which comprises an Uncaria rhynchophylla material.

The pharmaceutical composition of the present invention comprising the Uncaria rhynchophylla material can further selectively comprise a Cinnamomum cassia Presl material. Preferably, the content of the Uncaria rhynchophylla material is 30-80% by weight, and the content of the Cinnamomum cassia Presl material is 70-20% by weight.

Furthermore, the pharmaceutical composition of the present invention comprising the Uncaria rhynchophylla material can further comprise a composition consisting of an Ephedra material, a Prunus armeniaca L. var. ansu Maxim material, and a Glycyrrhiza uralensis Fisch. material. Preferably, the Ephedra material is selected from the group consisting of an Ephedra sinica Stapf material, an Ephedra equisetina Bge material, and an Ephedra intermedia Schrenk et.Mey material. Preferably, the content of the Uncaria rhynchophylla material is 10-40% by weight, the content of the Ephedra material is 15-45% by weight, the content of the Prunus armeniaca L. var. ansu Maxim material is 15%-45% by weight, and the content of the Glycyrrhiza uralensis Fisch material is 10%-30% by weight. The above-mentioned pharmaceutical composition can further comprise gypsum. Preferably, the content of gypsum is 20-80% by weight.

The present invention also provides a method for preparing the pharmaceutical composition for inhibiting snoring, comprising: providing an Uncaria rhynchophylla material.

In the above-mentioned method, a Cinnamomum cassia Presl material can be further provided to mix with the Uncaria rhynchophylla material. Preferably, the content of the Uncaria rhynchophylla material is 30-80% by weight, and the content of the Cinnamomum cassia Presl material is 70-20% by weight.

Alternatively, a composition consisting of an Ephedra material, a Prunus armeniaca L. var. ansu Maxim material, and a Glycyrrhiza uralensis Fisch. material is provided to mix with the Uncaria rhynchophylla material to form the pharmaceutical composition. Preferably, the content of the Uncaria rhynchophylla material is 10-40% by weight, the content of the Ephedra material is 15-45% by weight, the content of the Prunus armeniaca L. var. ansu Maxim material is 15%-45% by weight, and the content of the Glycyrrhiza uralensis Fisch material is 10%-30% by weight. The above-mentioned composition can be selectively mixed with gypsum. Preferably, the content of gypsum is 20-80% by weight.

The aforementioned “material” can be a Chinese traditional herb, the extract thereof, or the mixture of a Chinese traditional herb and the extract thereof. In addition, the extract of the herb can be provided by water or alcohol extraction.

Preferably, the materials of the pharmaceutical composition according to the present invention are extracts of herbs.

Preferably, the method for preparing the pharmaceutical composition of the present invention can further comprise: providing an excipient. If necessary, other pharmaceutical acceptable carriers can be further added. For example, they can be used in orally administered pharmaceutical composition, and the formulations include: capsules, tablets, emulsifiers, liquid suspensions, dispersants, and solvents. Taking tablets as an illustration, the commonly used carriers are galactose or cornstarch; and lubricants and magnesium stearate are basic additives. When oral capsules are used, galactose and dried corn starch can act as effective diluting agents. Suitable sweetening agents, flavoring agent or coloring agent be added if necessary. Preferably, the pharmaceutical composition of the present invention can further comprise an excipient, which can be any conventional one, preferably corn starch. The pharmaceutical composition can be granulated by spray-drying or freeze-drying. Preferably, the pharmaceutical composition is an oral formulation.

The extracts in the pharmaceutical composition of the present invention can be prepared by any method known in the art, preferably, by the method disclosed in the present invention.

Other objects, advantages, and novel features of the invention will become more apparent from the following detailed description when taken in conjunction with the accompanying drawings.

### BRIEF DESCRIPTION OF THE DRAWINGS

none

### DETAILED DESCRIPTION OF THE PREFERRED EMBODIMENT

The present invention provides an Uncaria rhynchophylla material and the pharmaceutical composition thereof for improving or inhibiting animal or human snoring. By conventional methods of preparation for Scientifically Concentrated Traditional Chinese Medicines including extraction, separation, concentration, and dryness (as disclosed in Sun-Tien Medical Journal 4 and 5, 2005), the extract powders of each herb were obtained and used on dogs like bulldogs, Beijing dogs, or Shih Tzu dogs. Due to their particular anatomical structures, these dog breeds have snoring syndromes like humans and thus they are used as subjects of experiments first.

Four pharmaceutical compositions provided in the present invention are concentrated powder capsules only containing an extract of an Uncaria rhynchophylla herb, concentrated powder capsules containing the composition A (shown in Table 1), concentrated powder capsules containing the composition A′ (shown in Table 2), and concentrated powder capsules containing the composition B (shown in Table 3), respectively.

TABLE 1
Composition A

| Material (Extract of Herb) | Weight(g) | Weight (%) |
|---|---|---|
| Uncaria rhynchophylla | 1.5~6 | 10~40 |
| Ephedra | 2.25~6.75 | 15~45 |
| Prunus armeniaca L. var. ansu Maxim | 2.25~6.75 | 15~45 |
| Glycyrrhiza uralensis Fisch. | 1.5~4.5 | 10~30 |

TABLE 2
Composition A′

| Material | Weight(g) | Weight % |
|---|---|---|
| Composition A | 3~12 | 20~80 |
| Gypsum | 12~3 | 80~20 |

TABLE 3
Composition B

| Material (Extract of Herb) | Weight(g) | Weight % |
|---|---|---|
| Uncaria rhynchophylla | 4.5~12 | 30~80 |
| Cinnamomum cassia Presl | 10.5~3 | 70~20 |

### EXAMPLES 1-17

Dogs having symptoms of snoring were selected, and administered with concentrated powder capsules only containing an extract of an Uncaria rhynchophylla herb (Examples 1-5), concentrated powder capsules containing the composition A (Examples 6-9), concentrated powder capsules containing the composition A′ (Examples 10 and 11), and concentrated powder capsules containing the composition B (Examples 12-17), respectively. The administered dose is based on body weight of the dogs, e.g., 1-2 g per 5 kg body weight per day. The results show great efficacy of inhibiting snoring, as shown in Table 4.

TABLE 4

| | Breed | | |
|---|---|---|---|
| | Weight (kg) | | |
| | Age (year) | Ingredient(s) in | |
| Example | Gender | capsules | Result |
| 1 | Shih tzu | Extract of Uncaria | After 5 days, snoring sounds were |
| | 1.7 | Rhynchophylla Herb | significantly down |
| | 3 | | |
| | Female | | |
| 2 | mongrel | Extract of Uncaria | After 3 days, snoring sounds were |
| | 12 | Rhynchophylla Herb | significantly down, and even disappeared |
| | 4 | | upon subsequent administration |
| | Male | | |
| 3 | Beijing | Extract of Uncaria | After 3 days, snoring sounds were |
| | 3 | Rhynchophylla Herb | significantly down, and even disappeared |
| | 5 | | upon subsequent administration |
| | Male | | |
| 4 | Pug | Extract of Uncaria | After 3-5 days, snoring sounds were |
| | 6 | Rhynchophylla Herb | significantly down |
| | 2.5 | | |
| | Male | | |
| 5 | Pug | Extract of Uncaria | After one week, snoring sounds were |
| | 4 | Rhynchophylla Herb | significantly down |
| | 6 | | |
| | Female | | |
| 6 | Beagle | Composition A | After 3 days, snores disappeared |
| | 10 | | |
| | 2 | | |
| | Male | | |
| 7 | Shih tzu | Composition A | Snoring sounds were down after |
| | 5 | | administration for 1 day, and disappeared |
| | 9 | | after 3 days. |
| | Male | | |
| 8 | Shih tzu | Composition A | Snoring sounds were down after |
| | 3 | | administration for 1 day, and disappeared |
| | 1.5 | | after 3 days. |
| | Female | | |
| 9 | Shih tzu | Composition A | After 5 days, snoring sounds were |
| | 4 | | significantly down |
| | 7 | | |
| | Feale | | |
| 10 | Pug | Composition A′ | After 2-3 days, snores disappeared |
| | 7 | | |
| | 8 | | |
| | Male | | |
| 11 | mongrel | Composition A′ | After 3 days, snoring sounds were |
| | 5 | | significantly down |
| | 5 | | |
| | Female | | |
| 12 | Bulldog | Composition B | After 5 days, snores disappeared |
| | 10 | | |
| | 5 | | |
| | Female | | |
| 13 | Beijing | Composition B | Snoring sounds were down after |
| | 5 | | administration for 1 day, and disappeared |
| | 8 | | after 5 days. |
| | Male | | |
| 14 | Shih tzu | Composition B | After 3 days, snores disappeared |
| | 4 | | |
| | 2 | | |
| | Male | | |
| 15 | Mongrel | Composition B | After 5 days, snores disappeared |
| | 9 | | |
| | 5 | | |
| | Female | | |
| 16 | Beijing | Composition B | After 3 days, snoring sounds were |
| | 3 | | significantly down |
| | 6 | | |
| | Male | | |
| 17 | Schnauzer | Composition B | After 7 days of administration followed by |
| | 2 | | cessation of administration, snores |
| | 5 | | disappeared and did not occur again after 30 |
| | Male | | days. |

The dogs in Examples 1-5 showed that snoring sounds diminished after 3-5 days of administering concentrated powder capsules containing an extract of an Uncaria rhynchophylla herb, and disappeared or were insignificant after 5-7 days. Thereby, the capsules containing the extract of the Uncaria rhynchophylla herb exhibits great efficacy of inhibiting snoring of dogs.

The dogs in Examples 6-9 showed that snoring sounds diminished or even disappeared after averagely 2-5 days of administering concentrated powder capsules containing the composition A. In addition, some dogs showed diminished snoring sounds even after only one day of administration, and thus it is indicated that the composition A exhibits efficient efficiency. Therefore, the capsules containing the composition A is advantageous over those only containing the extract of the Uncaria rhynchophylla herb in that the composition A exhibits efficient efficiency of inhibiting snoring of dogs. The dogs in Examples 10 and 11 showed similar responses after being treated with concentrated powder capsules containing the composition A′.

Though the composition A exhibits great efficacy in preventing snoring, the composition A is not suitable for some animals and humans, resulting from the extract of the Ephedra contained in the composition A. In view of this, the composition B (containing an extract of an Uncaria rhynchophylla herb and an extract of a Cinnamomum cassia herb) is developed.

The dogs in Examples 12-17 showed great responses of inhibition of snoring after being treated with the composition B for average 5 days. This composition does not contain the extract of the Ephedra, so it is milder than the composition A and has wider spectrum of use.

### EXAMPLES 18-41

The animal experiments illustrate the efficacy of the capsules containing the extract of the Uncaria rhynchophylla herb as the major content in improving and inhibiting animal snoring. Subsequently, the same compositions are used on humans. Concentrated powder capsules only containing the extract of the Uncaria rhynchophylla herb (Examples 18-21-24-27 and 30), capsules containing the composition A (Examples 19-22-25-28 and 31˜35), capsules containing the composition A′ (Examples 36 and 37), and capsules containing the composition B (Examples 20-23-26-29- and 38˜41) were administered. The administered dose for adults is 6-9 g/day, and 3-4.5 g/day for children. Obviously the subjects were unable by themselves to observe changes in their snoring, so observations from family members or tape-recorded snoring were used. The results are shown in Table 5.

TABLE 5

| | Age (year) | | | | |
|---|---|---|---|---|---|
| | Height (cm) | | | | |
| | Weight (kg) | | | | Special |
| | Sex | Ingredient(s) in | | | health |
| Example | Family Name | capsules | Condition of Snoring | Complications | history |
| 18 | 36 | Extract of Uncaria | Improved mildly after 3 | none | allergic |
| | 175 | Rhynchophylla | days | | rhinitis |
| | 65 | Herb | | | |
| 19 | Male | Composition A | Improved after 1 day, open | none | |
| | Lin | | airway, stopped snoring | | |
| | | | after 3 days | | |
| 20 | | Composition B | Improved mildly after 3 | none | |
| | | | days | | |
| 21 | 55 | Extract of Uncaria | Improved mildly | none | Long-term |
| | 172 | Rhynchophylla | | | smoking |
| | 70 | Herb | | | |
| 22 | Male | Composition A | Improved significantly | Palpitation | |
| 23 | Fei | Composition B | Great response, stopped | none | |
| | | | snoring by long-term | | |
| | | | administration (twice a | | |
| | | | week) | | |
| 24 | 53 | Extract of Uncaria | Improved mildly | none | Long-term |
| | 165 | Rhynchophylla | | | smoking |
| | 70 | Herb | | | |
| 25 | Male | Composition A | Improved significantly | dry mouth | |
| 26 | Yu | Composition B | Improved significantly, | none | |
| | | | relieved in the throat | | |
| 27 | 35 | Extract of Uncaria | Improved mildly | none | Long-term |
| | 167 | Rhynchophylla | | | fatigue |
| | 65 | Herb | | | |
| 28 | Male | Composition A | Improved | none | |
| 29 | Du | Composition B | Improved, relieved in the | none | |
| | | | throat | | |
| 30 | 55 | Extract of Uncaria | Improved mildly | none | Mild |
| | 175 | Rhynchophylla | | | rhinitis |
| | 68 | Herb | | | |
| 31 | Male | Composition A | Stopped snoring after 5 | none | |
| | Chen | | days, open airway | | |
| 32 | 38 | Composition A | Lowered snoring sounds | sleepy | Coffee |
| | 170 | | | | addiction |
| | 60 | | | | |
| | Male | | | | |
| | Hou | | | | |
| 33 | 55 | Composition A | Lowered snoring sounds | none | Obesity |
| | 149 | | and longer intervals | | |
| | 50 | | | | |
| | Female | | | | |
| | Wu | | | | |
| 34 | 45 | Composition A | No significant effects due to | none | Severe nasal |
| | 178 | | severe nasal disease | | disease |
| | 60 | | | | |
| | Male | | | | |
| | Wang | | | | |
| 35 | 7 | Composition A | No significant effects due to | none | Severe nasal |
| | 115 | | severe nasal disease | | disease |
| | 20 | | | | |
| | Male | | | | |
| | Luo | | | | |
| 36 | 10 | Composition A′ | Lowered or disappearing | none | allergic |
| | 155 | | snoring sounds after 3-5 | | rhinitis |
| | 28 | | days, stopped snoring by | | |
| | Male | | long-term administration | | |
| | Lin | | (twice a week) | | |
| 37 | 38 | Composition A′ | Improved sleep after | none | Mild |
| | 180 | | administration for 3 days | | rhinitis |
| | 78 | | | | |
| | Male | | | | |
| | Huang | | | | |
| 38 | 50 | Composition B | Lowered snoring sounds, | none | Obesity |
| | 158 | | improved concentration | | hypertension |
| | 97 | | during days and better sleep | | |
| | Female | | quality at night | | |
| | Zhu | | | | |
| 39 | 65 | Composition B | Great response, stopped | none | Long-term |
| | 172 | | snoring | | smoking |
| | 70 | | | | |
| | Male | | | | |
| | Chen | | | | |
| 40 | 40 | Composition B | Great response, stopped | none | Obesity |
| | 175 | | snoring | | |
| | 98 | | | | |
| | Male | | | | |
| | Lin | | | | |
| 41 | 50 | Composition B | Relieved in the throat, | none | Obesity |
| | 165 | | lowered snoring sounds | | |
| | 100 | | | | |
| | Male | | | | |
| | Wu | | | | |

The subjects in Examples 18, 21, 24, 27 and 30 showed lowered snoring sounds after taking the capsules only containing the extract of the Uncaria rhynchophylla herb for 3-5 days, and some of them even showed disappearing snoring. Thus, the capsules only containing the extract of the Uncaria rhynchophylla herb exhibits good efficacy in inhibiting human snoring for the subjects including rhinitis patients, long-term fatigue patients, and long-term smokers.

The subjects in Examples 19, 22, 25, 28 and 31-35 showed lowered or even disappearing snoring sounds averagely after taking the capsules containing the composition A for 3 days. Some of the subjects even showed lowered snoring sounds after administration for 1 day, and it is indicated that the effects are significant. The composition A employed to inhibit snoring in humans exhibits substantial efficacy for the subjects including rhinitis patients, long-term fatigue patients and long-term smokers. In addition, the subjects in Examples 19, 22, 25, 28 and 31-35 took not only the capsules containing only the extract of the Uncaria rhynchophyll herb but also the capsules containing the composition A. It was found that the composition A has better efficacy in mild rhinitis patients. Thus, it is shown that the composition A possesses superb efficacy in inhibiting snoring due to respiratory diseases. However, the efficacy for those having severe nasal diseases is not significant (see Examples 34 and 35), so they are not recommended to take the composition A. The capsules containing the composition A′ taken by the subjects in Examples 36 and 37 are provided for the subjects having “hot” constitution to attend the purposes of fever-clearing (resulting from gypsum contained in the composition A′).

For the subjects suffering from snoring not caused by respiratory diseases (e.g. obesity, long-term fatigue, and long-term smoking), the capsules containing the composition B were administered and great effects of inhibition of snoring were obtained. The subjects in Examples 20, 23, 26, 29, and 38-41 showed lowered snoring sounds averagely after 3 days of taking the capsules containing the composition B, and some of them even showed disappearing snoring. In Comparison to the conditions of taking the capsules only containing the extract of the Uncaria rhynchophyll herb on the same subject, the composition B exhibits more efficient efficiency and the patient taking the composition B felt more relieved in the throat (comparing Example 24 and 26, 27 and 29). The subjects in Examples 38-41 also showed great responses. The subject in the Example 38 is a patient suffering from obesity and hypertension, so she is not suitable for treatment with the composition A containing the extract of the Ephedra. Instead, the composition B would be more suitable.

To sum up, in view of animal and human clinical experiments, it is found that Uncaria rhynchophyll has significant effects in inhibiting or relieving snoring. For patients having mild rhinitis, the composition A gives better and more efficient effects. For snoring caused by non-respiratory diseases such as obesity, long-term smoking or fatigue, the composition B gives better effects.

What is claimed is:

1. A pharmaceutical composition for inhibiting snoring, comprising: an Uncaria rhynchophylla material.

2. The pharmaceutical composition of claim 1, wherein the Uncaria rhynchophylla material is an Uncaria rhynchophylla herb, an extract of an Uncaria rhynchophylla herb or the mixture thereof.

3. The pharmaceutical composition of claim 2, wherein the extract of the Uncaria rhynchophylla herb is provided by water or alcohol extraction.

4. The pharmaceutical composition of claim 1, further comprising an excipient.

5. The pharmaceutical composition of claim 1, wherein the pharmaceutical composition is an oral formulation.

6. The pharmaceutical composition of claim 1, further comprising a Cinnamomum cassia Presl material.

7. The pharmaceutical composition of claim 6, wherein the content of the Uncaria rhynchophylla material is 30-80% by weight, and the content of the Cinnamomum cassia Presl material is 70-20% by weight.

8. The pharmaceutical composition of claim 6, wherein the Cinnamomum cassia Presl material is a Cinnamomum cassia Presl herb, an extract of a Cinnamomum cassia Presl herb or the mixture thereof.

9. The pharmaceutical composition of claim 8, wherein the extract of the Cinnamomum cassia Presl herb is provided by water or alcohol extraction.

10. The pharmaceutical composition of claim 6, further comprising an excipient.

11. The pharmaceutical composition of claim 1, further comprising a composition consisting of an Ephedra material, a Prunus armeniaca L. var. ansu Maxim material, and a Glycyrrhiza uralensis Fisch. material.

12. The pharmaceutical composition of claim 11, wherein the Ephedra material is selected from the group consisting of an Ephedra sinica Stapf material, an Ephedra equisetina Bge material, and an Ephedra intermedia Schrenk et.Mey material.

13. The pharmaceutical composition of claim 11, wherein the content of the Uncaria rhynchophylla material is 10%-40% by weight, the content of the Ephedra material is 15-45% by weight, the content of the Prunus armeniaca L. var. ansu Maxim material is 15%-45% by weight, and the content of the Glycyrrhiza uralensis Fisch material is 10%-30% by weight.

14. The pharmaceutical composition of claim 11, wherein the composition is an herb mixture, an extract of an herb mixture or the mixture thereof.

15. The pharmaceutical composition of claim 14, wherein the extract of the herb mixture is provided by water or alcohol extraction.

16. The pharmaceutical composition of claim 11, further comprising an excipient.

17. The pharmaceutical composition of claim 11, further comprising gypsum.

18. The pharmaceutical composition of claim 17, wherein the content of the gypsum is 20-80% by weight.

19. A method for preparing a pharmaceutical composition for inhibiting snoring, comprising: providing an Uncaria rhynchophylla material.

20. The method of claim 19, wherein the Uncaria rhynchophylla material is an Uncaria rhynchophylla herb, an extract of an Uncaria rhynchophylla herb or the mixture thereof.

21. The method of claim 20, wherein the extract of the Uncaria rhynchophylla herb is provided by water or alcohol extraction.

22. The method of claim 20, further comprising: mixing the extract of the Uncaria rhynchophylla herb with an excipient and then granulation by spray-drying or freeze-drying.

23. The method of claim 19, wherein the pharmaceutical composition is an oral formulation.

24. The method of claim 19, further comprising: providing a Cinnamomum cassia Presl material and then mixing the Cinnamomum cassia Presl material with the Uncaria rhynchophylla material, wherein the content of the Uncaria rhynchophylla material is 30-80% by weight, and the content of the Cinnamomum cassia Presl material is 70-20% by weight.

25. The method of claim 24, wherein the Cinnamomum cassia Presl material is a Cinnamomum cassia Presl herb, an extract of a Cinnamomum cassia Presl herb or the mixture thereof.

26. The method of claim 25, wherein the extract of the Cinnamomum cassia Presl herb is provided by water or alcohol extraction.

27. The method of claim 25, further comprising: mixing the extract of the Uncaria rhynchophylla herb and the extract of the Cinnamomum cassia Presl herb with an excipient and then granulation by spray-drying or freeze-drying.

28. The method of claim 19, further comprising: providing a composition consisting of an Ephedra material, a Prunus armeniaca L. var. ansu Maxim material, and a Glycyrrhiza uralensis Fisch. material, and mixing the composition with the Uncaria rhynchophylla material, wherein the content of the Uncaria rhynchophylla material is 10-40% by weight, the content of the Ephedra material is 15-45% by weight, the content of the Prunus armeniaca L. var. ansu Maxim material is 15%-45% by weight, and the content of the Glycyrrhiza uralensis Fisch material is 10%-30% by weight.

29. The method of claim 28, further comprising gypsum, wherein the content of the gypsum is 20-80% by weight.

30. The method of claim 28, wherein the composition is an herb mixture, an extract of an herb mixture or the mixture thereof.

31. The method of claim 30, wherein the extract of the herb mixture is provided by water or alcohol extraction.

32. The method of claim 30, further comprising: mixing the Uncaria rhynchophylla material and the extract of the herb mixture with an excipient and then granulation by spray-drying or freeze-drying.
